(19) United States
(12) Patent Application Publication
(10) Pub. No.: US 2025/0110133 A1
(43) Pub. Date: Apr. 3, 2025

(54) METHOD AND KIT FOR PREDICTING DRUG EFFICACY OF LUNG CANCER TREATMENT AND METHOD FOR TREATING LUNG CANCER

(71) Applicants: National Health Research Institutes, Miaoli County (TW); National Taiwan University Hospital Hsin-Chu Branch, Hsinchu County (TW)

(72) Inventors: Chia-Huei Lee, Miaoli County (TW); Hsien-Neng Huang, Miaoli County (TW)

(73) Assignees: National Health Research Institutes, Miaoli County (TW); National Taiwan University Hospital Hsin-Chu Branch, Hsinchu County (TW)

(21) Appl. No.: 18/898,641

(22) Filed: Sep. 26, 2024

Related U.S. Application Data
(60) Provisional application No. 63/540,940, filed on Sep. 28, 2023.

(51) Int. Cl. G01N 33/574; A61K 31/404; A61K 31/506; A61K 31/517; A61K 31/5377; A61K 38/17; A61P 35/00

(52) U.S. Cl. CPC G01N 33/57423; A61K 31/404; A61K 31/506; A61K 31/517; A61K 31/5377; A61K 38/17; A61P 35/00

(57) ABSTRACT

Provided is a method for predicting drug efficacy of lung cancer, including providing a biological sample of a subject with lung cancer; analyzing an expression level of Leucine Zipper Down-regulated in Cancer 1; and predicting the drug efficacy based on the expression level of the Leucine Zipper Down-regulated in Cancer 1. Also provided is kit for predicting drug efficacy of lung cancer in a subject in need thereof, including an antibody against Leucine Zipper Down-regulated in Cancer 1 or a Leucine Zipper Down-regulated in Cancer 1-specific primer. Further provided is a method for treating lung cancer, including enhancing expression of Leucine Zipper Down-regulated in Cancer 1 encoded by Ldoc1 gene in a subject in need thereof.

### CROSS REFERENCE TO RELATED APPLICATIONS

This application claims the benefit of U.S. Provisional Application No. 63/540,940, filed on Sep. 28, 2023. The content of the application is incorporated herein by reference.

The present application hereby incorporates by reference the entire contents of the text file named “NHR-P0022-USA-Sequencing Listing.xml” in XML format. The text file containing the Sequencing Listing of the present application was created on Sep. 19, 2024 and is 3,881 bytes in size.

### BACKGROUND OF THE INVENTION

### 1. Field of the Invention

The present disclosure relates to a drug efficacy prediction technique and a method for treating lung cancer, and more particularly, to a drug efficacy prediction technique for predicting the drug efficacy of lung cancer treatment and a method for treating a non-small cell lung cancer.

### 2. Description of the Prior Art

The epidermal growth factor receptor-tyrosine kinase inhibitors (EGFR-TKIs) are particularly effective in non-small cell lung cancer (NSCLC) patients with mutant EGFR (EGFR^{M}). Therefore, tumor biopsies obtained from advanced NSCLC are firstly examined for EGFR genotype to assess whether to receive first-generation EGFR-TKI treatment, e.g., gefitinib (Iressa).

However, there are approximately 50% to 70% of advanced NSCLC patients with EGFR^{M} not showing responses (non-responders) to the first-generation EGFR-TKI treatment. Moreover, about 30% of the advanced NSCLC patients with EGFR^{M} who show responses (responders) to the first-generation EGFR-TKI treatment develop de novo drug resistance after three months of the treatment. Since health insurance coverage is limited, i.e., only EGFR-TKI treatment for NSCLC patients with EGFR^{M} is covered, other alternative therapeutic options/strategies are not considered typically, leading to delayed treatment.

Based on the above, there is an unmet need in the art to develop a more accurate detection method for identifying responder among EGFR^{M} NSCLC patients, and to monitor acquired resistance among EGFR^{M} patients receiving EGFR-TKI treatment.

### SUMMARY OF THE INVENTION

Other aspects of the present disclosure will be set forth in the description which follows, and in part will be obvious to one of ordinary skill in the art after perusing the following content. One of ordinary skill in the art may also conceive the content thereof from the implementation of the present disclosure. The advantages disclosed herein may be realized and obtained as particularly pointed out in the appended claims.

To solve the aforementioned problems, the present disclosure provides a method for predicting drug efficacy of lung cancer, including providing a biological sample of a subject with lung cancer; analyzing an expression level of Leucine Zipper Down-regulated in Cancer 1 (LDOC1); and predicting the drug efficacy based on the expression level of the Leucine Zipper Down-regulated in Cancer 1.

The present disclosure further provides a kit for predicting drug efficacy of lung cancer in a subject in need thereof, including an antibody against Leucine Zipper Down-regulated in Cancer 1 or a Leucine Zipper Down-regulated in Cancer 1-specific primer.

The present disclosure further provides a method for treating lung cancer, including enhancing expression Leucine Zipper Down-regulated in Cancer 1 (LDOC1) encoded by Ldoc1 gene in a subject in need thereof.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram illustrating an exemplifying workflow of a method for predicting drug efficacy of lung cancer according to an embodiment of the present disclosure.

FIG. 2A is a sequence illustrating human LDOC1 according to an embodiment of the present disclosure. Open box indicates adaptin binding motifs clathrin adaptor proteins in human LDOC1.

FIG. 2B is images of co-immunoprecipitation and western blot analysis revealing endogenous LDOC1-AP1M1 and LDOC1-AP2M1 interactions in A549 cells according to an embodiment of the present disclosure. Cells are serum-starved overnight and then subjected to 10 nM EGF stimulation for 30 min. Protein lysates are then harvested and subjected to co-immunoprecipitation using antibodies against LDOC1, AP1M1, or AP2M1, followed by western blot analysis using antibodies as indicated. “Input” indicates total cellular protein lysates; “IgG” indicates immunoglobulin G; and “a” denotes antibodies specifically against the indicated target protein of the present disclosure.

FIG. 2C is images of co-immunoprecipitation and Western blot analysis revealing endogenous LDOC1-AP1M1 and LDOC1-AP2M1 interactions in PC9 cells according to an embodiment of the present disclosure. Cells are serum-starved overnight and then subjected to 10 nM EGF stimulation for 30 min. Protein lysates are then harvested and subjected to co-immunoprecipitation using antibodies against LDOC1, AP1M1, or AP2M1, followed by Western blot analysis using antibodies as indicated. “Input” indicates total cellular protein lysates; “IgG” indicates immunoglobulin G; and “a” denotes antibodies specifically against the indicated target protein of the present disclosure.

FIG. 2D is images of double immunofluorescence staining analysis indicating the interactions between exogenous LDOC1 and AP1M1 according to an embodiment of the present disclosure. Plasmids expressing LDOC1-V5 with AP1M1-FLAG are co-transfected into U2OS cells, which are serum-starved for 6 h on the next day and then subjected to EGF (10 nM) stimulation. After EGF stimulation for 30 min, immunostaining is conducted using anti-V5 (red) and anti-FLAG (green) antibodies 24 h after transfection. Boxed regions are magnified and presented on the right. Colocalization of LDOC1-V5 and AP1M1-FLAG are presented as white speckles. Images are captured using a confocal microscope (Leica TCS SP5 II). Scale bar, 10 μm.

FIG. 2E is images of double immunofluorescence staining analysis indicating the interactions between exogenous LDOC1 and AP2M1 according to an embodiment of the present disclosure. Plasmids expressing LDOC1-V5 with AP1M1-FLAG or AP2M1-FLAG were co-transfected into U2OS cells, which were serum-starved for 6 h on the next day and then subjected to EGF (10 nM) stimulation. After EGF stimulation for 30 min, immunostaining was conducted using anti-V5 (red) and anti-FLAG (green) antibodies 24 h after transfection. Boxed regions are magnified and presented on the right. Colocalization of LDOC1-V5 and AP2M1-FLAG are presented as white speckles. Images are captured using a confocal microscope (Leica TCS SP5 II). Scale bar, 10 μm.

FIG. 3A is a schematic diagram illustrating EGFR endocytosis assay completed using cell surface labelling method according to an embodiment of the present disclosure. Serum starved cells were labeled with sulfo-NHS-SS-biotin and incubated with Dulbecco's modified Eagle's medium (DMEM) containing EGF (10 nM) for 10 min at 37° C. for internalization (In). Cells were then treated with membrane-impermeable reducing reagent to remove biotin from proteins on the plasma membrane (first stripping, 1s). Cells were re-incubated with DMEM at 37° C. for 30 min to allow for the recycling of internalized biotinylated proteins (Re) and then treated with reducing reagent (second stripping, 2s).

FIG. 3B is images illustrating treatment of LDOC1-depleted (shLDOC1) and control (shCtrl) HCC827 (left panel) and PC9 (right panel) cells according to an embodiment of the present disclosure. After internalization (In), first stripping (1s), recycling (Re), and second stripping (2s), whole-cell lysates are prepared (total), and biotinylated proteins are collected using an avidin column (pulldown).

FIG. 3C is bar charts illustrating treatment of LDOC1-depleted (shLDOC1) and control (shCtrl) HCC827 (upper left and lower left panel) and PC9 (upper right and lower right panel) cells according to an embodiment of the present disclosure. After internalization (In), first stripping (1s), recycling (Re), and second stripping (2s), whole-cell lysates were prepared (total), and biotinylated proteins were collected using an avidin column (pulldown). Subsequently, western blot analysis is conducted using the antibodies of the present disclosure. Immunoblots are quantified using ImageJ (version 1.47). Ratios of internalization (1s/In) and recycling [(Re−2s)/Re] are calculated and plotted as bar charts. Data are presented as mean±SEM, n=3. Differences between shCtrl and shLDOC1 cells are analyzed using Student's t test. “pEGFR” indicates phosphorylated EGFR; and “tEGFR” indicates total EGFR.

FIG. 4 is images illustrating western blotting of HCC827 (left panel) and PC9 (right panel) cells with (shLDOC1) and without (shCtrl) LDOC1 depletion according to an embodiment of the present disclosure. After biotinylation of surface proteins, cellular protein lysates (total) and the plasma membrane (PM) fraction are analyzed using antibodies against phosphorylated and total AXL, HER2, and HER3. Five percent of the input is applied in the total lane. Statistical differences between each shLDOC1 and shCtrl are analyzed using Student's t-test.

Left panels of FIG. 5 are histopathological sections illustrating that LDOC1 depletion is associated with diffuse cytoplasmic EGFR staining in EGFR^{WT} (upper left panel) and EGFR^{M} (lower left panel) NSCLC tumors according to an embodiment of the present disclosure. Scale bar=50 μm.

Right panels of FIG. 5 are Kaplan-Meier survival curves for patients with advanced EGFR^{WT} (upper right panel) or EGFRM (lower right panel) NSCLC stratified into membranous (memb.) and cytoplasmic (cyto.) EGFR according to an embodiment of the present disclosure.

FIG. 6A is images illustrating that LDOC1 depletion upregulated expression of phosphorylated (p) and total (t) EGFR and related RTKs thereof according to an embodiment of the present disclosure. PC9 cells are serum-starved for 16 h before being treated with EGF (10 nM) for 30 min at 37° C. Whole-cell lysates are subjected to Western blot analysis using the indicated antibodies of the present disclosure. GAPDH is used as a loading control.

FIG. 6B is images illustrating that LDOC1 depletion upregulated expression of phosphorylated (p) and total (t) EGFR and related RTKs thereof according to an embodiment of the present disclosure. HCC827 cells are serum-starved for 16 h before being treated with EGF (10 nM) for 30 min at 37° C. Whole-cell lysates are subjected to western blot analysis using the indicated antibodies of the present disclosure. GAPDH is used as a loading control.

FIG. 6C is images illustrating that LDOC1 depletion sustained prolonged activation of EGFR, HER2, and AXL according to an embodiment of the present disclosure. PC9 cells are serum-starved overnight and then treated with EGF (10 nM) at 37° C. for 10 min, washed with PBS, and then incubated in normal growth medium at 37° C. The value of pRTK/GAPDH at time 0 was set as 1 and fold changes >1 are shown in italics.

FIG. 6D is images illustrating that semiquantitative evaluation of FIG. 6C according to an embodiment of the present disclosure. The average results are plotted using arbitrary unit (a.u.) for pEGFR, pHER2, pHER3, and pAXL.

Upper panel of FIG. 7A is images illustrating effects of LDOC1 depletion on IC50 for gefitinib, erlotinib, and osimertinib in PC9 cells determined by using MTT assays according to an embodiment of the present disclosure. Cells were seeded in 96-well cell culture plates and treated the next day with the indicated concentrations of indicated EGFR-TKI. After 3 days, cell numbers were determined with MTT assay.

Lower panel of FIG. 7A is images illustrating effects of LDOC1 depletion on IC50 for gefitinib, erlotinib, and osimertinib in HCC827 cells determined by using MTT assays according to an embodiment of the present disclosure.

Left panel of FIG. 7B is images illustrating effects of LDOC1 depletion on IC50 for gefitinib, erlotinib, and osimertinib in PC9 cells determined by using colony-forming assays according to an embodiment of the present disclosure. Data are presented as mean±standard error of mean, n=3. Differences between shLDOC1 and shCtrl at each indicated concentration are analyzed using Student's t test; * p<0.05 and ** p<0.01.

Right panel of FIG. 7B is images illustrating Kaplan-Meier survival curves for patients with advanced EGFR^{M} NSCLC receiving gefitinib, stratified into high (n=58) and low (n=42) LDOC1 expression according to an embodiment of the present disclosure.

### DETAILED DESCRIPTION

The following descriptions of the embodiments illustrate implementations of the present disclosure, and those skilled in the art of the present disclosure can readily understand the advantages and effects of the present disclosure in accordance with the contents herein. However, the embodiments of the present disclosure are not intended to limit the scope of the present disclosure. The present disclosure can be practiced or applied by other alternative embodiments, and every detail included in the present disclosure can be changed or modified in accordance with different aspects and applications without departing from the essentiality of the present disclosure.

As used herein, the term “administering” may refer to the placement of an active ingredient (e.g., Leucine Zipper Down-regulated in Cancer 1 (LDOC1) protein sequence-derived polypeptide, or a LDOC1 gene sequence-derived DNA or RNA) into a subject by a method or appropriated route, systemically or locally, that results in at least partial localization of the active ingredient at a desired site to produce the desired effect. For example, the active ingredient of the present disclosure may be administered to the subject by subcutaneous administration, intracutaneous administration, intravenous administration, intramuscular administration, intraarticular administration, intraarterial administration, intrasynovial administration, intrasternal administration, intrathecal administration, intralesional administration, and intracranial administration or infusion techniques, but the present disclosure is not limited thereto.

As used herein, the term “treat,” “treating,” or “treatment” refers to obtaining a desired pharmacologic and/or physiologic effect. The effect may be prophylactic in terms of completely or partially preventing a disease or symptoms or conditions thereof or may be therapeutic in terms of completely or partially curing, alleviating, relieving, remedying, or ameliorating a disease or an adverse effect attributable to the disease or symptoms or conditions thereof.

As used herein, the term “subject” may include mammalian and non-mammalian animals. The mammalian animals may include, but are not limited to, humans, non-human primates, canines, felines, murine, bovines, equines, pigs, sheep, deer, wolfs, foxes, and rabbits. The non-mammalian animals may include, but are not limited to, class Aves (e.g., birds or chickens) and fishes.

Unless otherwise specified, the terms “subject” and “patient” used herein are interchangeable.

As used herein, “comprising” (and any variant or conjugation thereof, such as “comprise” or “comprises”), “including” (and any variant or conjugation thereof, such as “include” or “includes”), or “having” (and any variant or conjugation thereof, such as “have” or “has”) a specific element, unless otherwise specified, may include other elements such as steps, components, ingredients, regions, portions, compositions, or domains rather than exclude those elements.

As used herein, the phrase “at least one,” in reference to a list of one or more elements, should be understood to mean at least one element selected from any one or more of the elements in the list of elements, but not necessarily including at least one of each and every element listed within the list of elements and not excluding any combinations of elements in the list of elements. This definition also allows that elements may optionally be present other than the elements identified within the list of elements to which the phrase “at least one” refers, whether related or unrelated to those elements identified. Thus, as a non-limiting example, “at least one of A and B” (or, equivalently, “at least one of A or B,” or, equivalently, “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

In addition, unless otherwise specified, the singular forms “a/an” and “the” used herein also include plural forms, and the terms “or” and “and/or” used herein are interchangeable.

Unless otherwise specified, the terms “mutant EGFR,” “EGFR-mutated,” and “EGFR^{M}” used herein are interchangeable.

Unless otherwise specified, terms “treatment” and “therapy” used herein may be used interchangeably.

Unless otherwise specified, terms “specimens” and “biological sample” used herein may be used interchangeably.

As used herein, “cell” refers to the smallest structural unit of living matter capable of functioning autonomously, consisting of one or more nuclei, cytoplasm, and various organelles, all surrounded by a semipermeable membrane. Cells include all somatic cells obtained or derived from a living or deceased animal body at any stage of development as well as germ cells, including sperm and eggs (animal reproductive body consisting of an ovum or embryo together with nutritive and protective envelopes). Included are both general categories of cells: prokaryotes and eukaryotes. The cells contemplated for use in this invention include all types of cells from all organisms in all kingdoms: plans, animals, protists, fungi, archaebacteria and eubacteria.

The numeral ranges used herein are inclusive and combinable, any numeral value that falls within the numeral scope herein could be taken as a maximum or minimum value to derive the sub-ranges therefrom. For example, the numeral range “0.9 to 1.3” includes any sub-ranges between the minimum value of 0.9 to the maximum value of 1.3, such as the sub-ranges from 0.9 to 1.1, from 1.1 to 1.3 μM, from 1.0 to 1.2 μM and so on. In addition, a plurality of numeral values used herein can be optionally selected as maximum and minimum values to derive numerical ranges. For instance, the numerical ranges of 0.9 to 1.1, 1.1 to 1.3, and 0.9 to 1.3 can be derived from the numeral values of 0.9, 1.1, and 1.3.

In at least one embodiment of the present disclosure, the drug efficacy may be an efficacy of the epidermal growth factor receptor-tyrosine kinase inhibitor (EGFR-TKI) on the subject.

In at least one embodiment of the present disclosure, the EGFR-TKI may be at least one selected from the group consisting of gefitinib, erlotinib, osimertinib, afatinib, and sunitinib.

In at least one embodiment of the present disclosure, the subject has not been administered by the epidermal growth factor receptor-tyrosine kinase inhibitor.

In at least one embodiment of the present disclosure, the method for predicting drug efficacy of lung cancer may further include administering the epidermal growth factor receptor-tyrosine kinase inhibitor to the subject, provided that the expression level of Leucine Zipper Down-regulated in Cancer 1 (LDOC1) shows an increased level.

In at least one embodiment of the present disclosure, the method for predicting drug efficacy of lung cancer may further include providing the subject with an alternative treatment other than administration of the epidermal growth factor receptor-tyrosine kinase inhibitor, provided that the expression level of Leucine Zipper Down-regulated in Cancer 1 (LDOC1) shows a decreased level.

In at least one embodiment of the present disclosure, the lung cancer may be a non-small cell lung cancer (NSCLC).

In at least one embodiment of the present disclosure, the lung cancer may be EGFR-mutated (EGFR^{M}) lung cancer.

In at least one embodiment of the present disclosure, the kit may further include an experimental protocol of LDOC1 immunohistochemistry (IHC) testing, IHC reagents, IHC images for scoring the IHC staining intensities, and/or a protocol of quantitative RT-PCR.

In at least one embodiment of the present disclosure, the method for treating lung cancer may increase susceptibility of the subject to the epidermal growth factor receptor-tyrosine kinase inhibitor (EGFR-TKI).

In at least one embodiment of the present disclosure, the method for treating lung cancer may further include administering the subject with a Leucine Zipper Down-regulated in Cancer 1 (LDOC1) protein sequence-derived polypeptide to enhance the Leucine Zipper Down-regulated in Cancer 1 (LDOC1) expression in the subject.

In at least one embodiment of the present disclosure, the method for treating lung cancer may further include administering the subject with a Ldoc1 gene sequence-derived nucleotide to enhance the Leucine Zipper Down-regulated in Cancer 1 (LDOC1) expression in the subject.

In some embodiments of the present disclosure, LDOC1 depletion promotes internalization of EGFR and sustains enhanced and prolonged EGFR signaling and the activation of AXL, HER2, and HER3 in EGFR^{M} NSCLC PC9 and HCC827 cell lines. The sensitivity of EGFR^{M} NSCLC PC9 and HCC827 cells to EGFR-TKI (e.g., gefitinib and erlotinib) is significantly reduced by LDOC1 depletion. Low expression of LDOC1 strongly associates with poor overall survival in patients with advanced EGFR^{M} NSCLC receiving EGFR-TKI treatment (n=100, P<0.001). Accordingly, LDOC1 depletion may hinder the effective targeting EGFR-TKIs to EGFR, sustain EGFR signaling, and trigger acquired resistance to EGFR-TKI in LDOC1 negative (−) EGFR^{M} NSCLC cells, and is thus less susceptible to first- and second-generation EGFR-TKI (e.g., gefitinib, erlotinib, and sunitinib). Therefore, patients with EGFR^{M} NSCLC can be stratified by LDOC1 expression levels into responder (high expression of LDOC1) and non-responder (low expression of LDOC1).

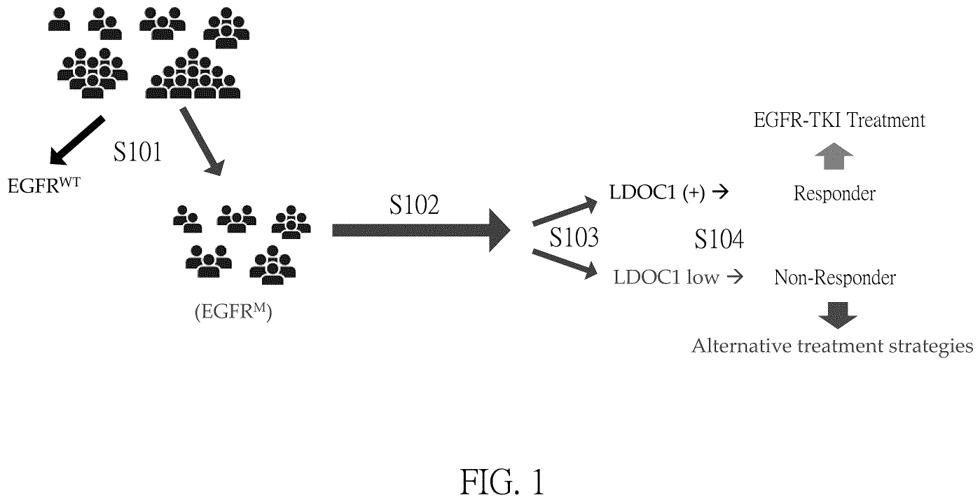

Referring to FIG. 1, a method for predicting drug efficacy of lung cancer is illustrated, and the operation process is denoted as arrows (described as “step(s)” herein) and explained from here.

In some embodiments, step 101 (S101) denotes that subjects with lung cancer (e.g., NSCLC) having EGFR^{M} are recruited for predicting drug (e.g., EGFR-TKI) efficacy of lung cancer. As shown in FIG. 1, “EGFR^{WT}” indicates wide-type EGFR.

In some embodiments, step 102 (S102) denotes that a biological sample of the subjects with lung cancer are collected and an expression level of LDOC1 may be examined by immunohistochemical (IHC) staining or quantitative real-time PCR, but the present disclosure is not limited thereto. For example, anti-LDOC1 antibodies are used to examine the LDOC1 protein expression in the biological sample (e.g., EGFR^{M} NSCLC specimens) by IHC staining analysis; or RNA collected from the circulating tumor cells of the subjects with lung cancer (e.g., NSCLC) and extracted from the biological sample (e.g., EGFR^{M} NSCLC tumors) is subject to carry out reverse transcription reaction and quantitative real-time PCR with LDOC1 primer pairs to examine the expression of LDOC1 mRNA.

In some embodiments, step 103 (S103) and step 104 (S104) denote that subjects with high expression of LDOC1 are expected to benefit from EGFR-TKI treatment (e.g., gefitinib treatment), while those with low expression of LDOC1 are recommended to treat with other anti-cancer agents or to seek for alternative treatment strategies. As shown in FIG. 1, “LDOC1 (+)” indicates high expression of LDOC1 or LDOC1 positive; and “LDOC1 low” indicates low expression of LDOC1 or LDOC1 negative.

In some embodiments of the present disclosure, LDOC1 may function as a prognostic biomarker for EGFR^{M} NSCLC and may regulate the efficacy of first- and second-generation EGFR-TKIs; and the underlying mechanism is illustrated below.

Non-small cell lung cancer (NSCLC) is responsible for 75% to 80% of all lung cancers and is a leading cause of cancer-related mortality. The epidermal growth factor receptor (EGFR), which belongs to the ErbB family, is the most common oncogenic driver for EGFR-mutated (EGFR^{M}) NSCLC. Clinical trials have demonstrated that EGFR-tyrosine kinase inhibitor (EGFR-TKI) therapy is highly effective for treating EGFR^{M} NSCLC, resulting in high response rates and improved survival. Mutations in the EGFR gene, including a small in-frame deletion in exon 19 and L858R point mutation in exon 21, accounted for 85% to 95% of EGFR^{M} NSCLC patients who responded to EGFR-TKIs. These recurrent mutations are mapped onto the encoding region for the receptor tyrosine kinase (RTK) domain, which results in an increased sensitivity to exogenous growth factors, leading to sustained activation of EGFR signaling in tumors. These mutations are therefore referred to as EGFR-activating mutations. NSCLC harboring EGFR^{M} becomes highly dependent on the EGFR signaling pathway for growth, exhibiting an “oncogenic addiction” to the pathway. First-generation EGFR-TKIs, such as gefitinib and erlotinib, reversibly bind to the ATP-binding pocket within the tyrosine kinase domain of EGFR. This blockage hinders cell proliferation and ultimately leads to cell death. EGFR^{M} exhibits greater affinity for gefitinib and erlotinib than does the wild-type version, making patients harboring EGFR^{M} are more responsive to EGFR-TKI therapy. Because of the higher response rates (50% to 70% after initial treatment) to gefitinib and erlotinib treatment among patients with EGFR^{M} than among those with EGFR^{WT}, gefitinib and erlotinib are approved for the first-line treatment of EGFR^{M} NSCLC patients. However, the efficacy of gefitinib or erlotinib has been limited by intrinsic and acquired resistance.

AXL RTK is a member of the TAM (TYRO3/AXL/MERTK) RTK family and plays a key role in intrinsic and acquired resistance to gefitinib and erlotinib in EGFR^{M} NSCLC. In at least one embodiment of the present application, AXL may amplify EGFR signaling by interacting with EGFR in brain tumor cells. The most noteworthy observation to date is that AXL is upregulated by EGFR-TKIs and induces endogenous mutators, such as components involving error-prone DNA replication, and drives the transition from drug-tolerant persister to resister in EGFR^{M} NSCLC cells. Amplification of other members of the ErbB gene family, such as ErbB2 (HER2) and ErbB3 (HER3), has also been implicated in resistance to EGFR-TKIs. Exogenous expression of HER2 was reported to be associated with reduced sensitivity to third-generation EGFR-TKIs in EGFR^{M} NSCLC PC9 cells, indicating that HER2 activity underlies the resistance of EGFR^{M} NSCLC cells to EGFR-TKIs. Additionally, observations from clinical samples revealed that it augmented HER3 in EGFR^{M} NSCLC tumors with acquired EGFR-TKI resistance. In at least one embodiment of the present application, it is believed that identification of factors influencing activation and expression of AXL and members of the ErbB family may be useful for improving the efficacy of EGFR-TKIs and may thereby benefit patients with EGFR^{M} NSCLC.

EGFR binds to ligands at its extracellular ligand-binding domains, promoting homo- and hetero-dimerization with other members of the ErbB family, thereby activating intracellular TK domains thereof. This process induces the activation of downstream signaling pathways and components involved in cell proliferation, cell cycle progression, viability, and motility. Signal transduction of RTKs is regulated by endocytosis. The endocytic pathway can be either clathrin-dependent or independent. Clathrin-mediated endocytosis (CME) plays a crucial role in modulating EGFR signaling and is the primary pathway for EGFR internalization. While it is widely accepted that CME attenuates RTK signaling, accumulating evidence suggests that blocking internalization also reduces some RTK signaling, including EGFR. In at least one embodiment of the present application, it is demonstrated that EGFR internalized through CME is not targeted for lysosomal degradation but is instead recycled to the plasma membrane (PM). Therefore, clathrin-mediated internalization (CMI), rather than receptor degradation, is essential for sustained EGFR signaling. In CME, the clathrin adaptors AP-1 and AP-2 connect membranes and cargo to a clathrin scaffold, controlling the internalization and recycling of specific membrane proteins, including EGFR. Adaptor proteins are central to CME. AP-1 and AP-2 are heterotetrameric complexes, with three of their four subunits sharing primary and ternary structural homologies: the large-chain β1 (AP1B1) and β2 (AP2B1), medium-chain μ1A (AP1M1) and μ2 (AP2M1), and small-chain σ1 (AP1S1) and σ2 (AP2S1). The fourth subunits, tγ1-adaptin of AP-1 (AP1G1) and α-adaptin of AP-2 (AP2A1), exhibit less sequence homology but have nearly identical ternary structures. AP-1 mediates vesicle protein sorting between the trans-Golgi network and endosomes. Ablation of AP-1 suppresses EGFR recycling to the PM, regardless of epidermal growth factor (EGF) stimulation, suggesting that AP-1's role in recycling endosomes is to retrieve internalized EGFR to maintain cell surface expression. AP-2, the most abundant clathrin adaptor in mammalian cells, is involved in the early phases of clathrin-coated pit nucleation and maturation, as well as the internalization of membrane proteins. It recruits cargo as clathrin-coated pit curvature increases and matures. This step serves as an “endocytic checkpoint” because failure to recruit cargo results in transient, abortive clathrin coated pits. During endocytosis, AP-1 and AP-2 shuttle between the PM and the trans-Golgi network with clathrin. Most membrane proteins undergoing CME are selected through the recognition of short linear amino acid motifs in the cytoplasmic domain thereof by clathrin adaptors, such as the subunits of AP-1 and AP-2. The two major classes of peptide motifs recognized by clathrin adaptors are the tyrosine-based YXXφ (where X stands for any amino acid and φ stands for an amino acid with a bulky hydrophobic side chain) and the acidic dileucine motif [E/D]xxxL[LI]. These sequence motifs, also known as endocytic codes, function as address tags that facilitate the delivery of transmembrane proteins and the effective targeting of endocytic machinery. Activated EGFR is internalized more rapidly through CME than in clathrin-independent endocytosis. Under particular physiological conditions, such as high ligand concentration and elevated reactive oxygen species production, EGFR internalization may occur through clathrin independent endocytosis, where the receptor is taken up by caveolae or through macropinocytosis.

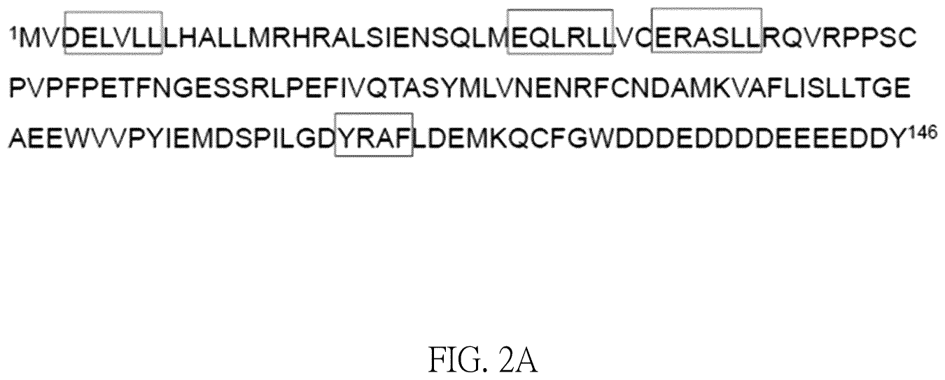

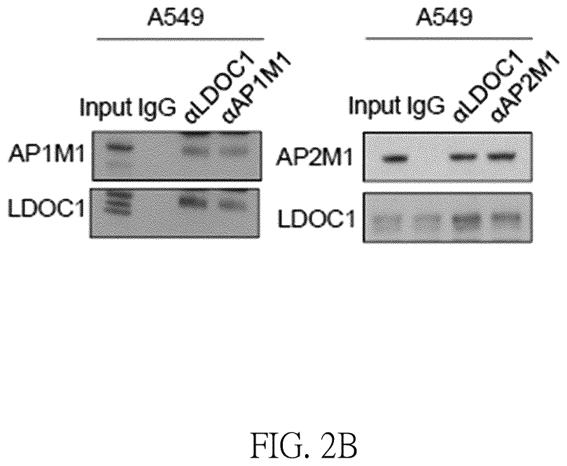

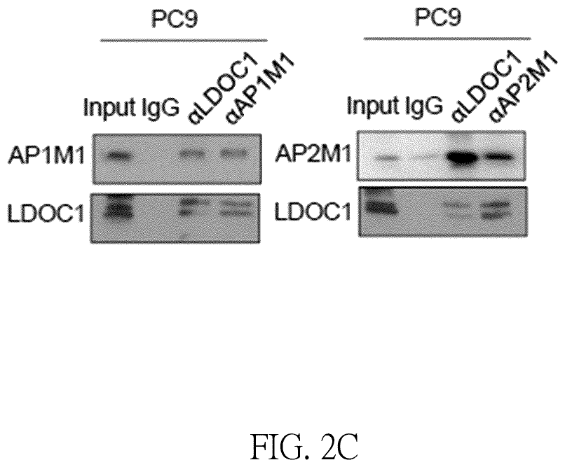

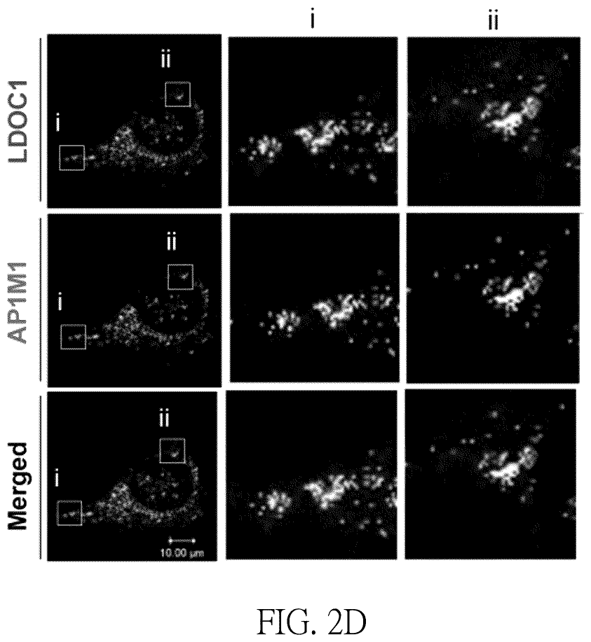

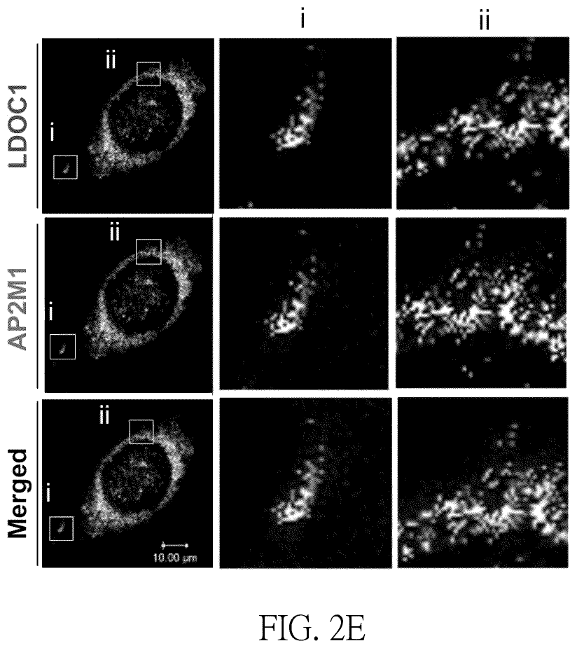

The LDOC1 gene encodes Leucine Zipper Down-regulated in Cancer 1, a 146-amino-acid protein (SEQ ID NO. 1), with an N-terminal leucine zipper motif and a proline-rich region similar to an SH3-binding domain (Uniprot accession number: 095751). AP1M1 may interact with LDOC1. Sequence analysis revealed multiple clathrin adaptor-binding motifs, including three highly conserved acidic dileucine motifs and one tyrosine-based motif, in the leucine zipper region and C-terminal domain of LDOC1, respectively (FIG. 2A). In at least one embodiment of the present application, LDOC1 functions as a tumor suppressor gene in two EGFR-driven cancers: oral squamous cell carcinoma and NSCLC. However, the present disclosure is not limited to these cancers. LDOC1 may interact with clathrin adaptors and regulate the CMI of EGFR. Additionally, LDOC1 may affect the internalization and activation of AXL, HER2, and HER3, as these molecules may bind to activated EGFR to form heterodimer complexes in NSCLC. In at least one embodiment of the present application, LDOC1 functions as a central regulator of EGFR internalization in EGFR^{M} NSCLC, partly due to its interaction with clathrin adaptors. However, the present disclosure is not limited to this function. LDOC1 depletion enhanced the activation of EGFR, HER2, HER3, and AXL. Furthermore, LDOC1 downregulation is strongly correlated with poor overall survival in patients with EGFR^{M} advanced NSCLC who received EGFR-TKI (e.g., gefitinib).

### Materials and Methods

### Patients and Tumor Biopsies

All specimens and clinical data are collected from patients who undergo a bronchoscopic biopsy, transthoracic biopsy, or surgery at National Taiwan University Hospital Hsin-Chu Branch. Formalin-fixed, paraffin-embedded tissue specimens acquired from 2012 to 2020 are obtained from the archives of Department of Pathology, National Taiwan University Hospital. The 200 patients included in the present application have advanced NSCLC, including 100 patients with EGFR^{M} and 100 patients with EGFR^{WT} who undergo gefitinib treatment and chemotherapy, respectively. The follow-up period ranged from 0.4 months to 70.5 months. Pathological sections stained with hematoxylin and eosin (HE) are reviewed and are used for the diagnosis of lung adenocarcinoma or NSCLC as World Health Organization classifications. This application of the present disclosure is approved by the Research Ethics Committee B, National Taiwan University Hospital, Taiwan (202303103RINB). Informed consent is obtained from each patient.

### Cell Culture, EGF Stimulation, and Reagents

A549, PC9, and U2OS cell lines are obtained from the American Type Culture Collection (Manassas, VA, USA). The HCC827 cell line is provided by Dr. Yi-Rong Chen from the Institute of Molecular and Genomic Medicine, National Health Research Institutes, Taiwan. A549 cells are maintained in DMEM/high glucose (Cytiva, Marlborough, MA, USA). PC9 and HCC827 cells are maintained in RPMI1640 (Cytiva). The U2OS cell line is cultured in McCoy's 5A (Modified) Medium (Gibco, Billings, MT, USA). All cells are cultured in media containing 10% FBS (Gibco), 1% penicillin (Thermo Fisher, Waltham, MA, USA), and 1% streptomycin (Thermo Fisher); the cells are incubated at 37° C. in a humidified chamber with 5% CO2. All cells that stably expressed siRNA-targeting LDOC1 or the scramble controls are maintained in growth medium containing 5 μg/mL puromycin (InvivoGen, San Diego, CA, USA). The cells are checked for Mycoplasma (BioSmart, Seoul, Republic of Korea) upon thawing, and throughout the experimental period they consistently tested negative for Mycoplasma. The cells are starved in serum-free medium overnight before EGF (10 nM) stimulation for the endocytosis assay, kinetics analysis of EGF-induced EGFR phosphorylation, and immunoprecipitation-western blot analysis. For immunofluorescence staining, the cells are grown overnight in medium containing 0.05% FBS before EGF stimulation. Gefitinib is purchased from Selleckchem (Houston, TX, USA), and 3CAI and ASTX029 are purchased from MedChemExpress (New York, NJ, USA).

### Plasmid Construction, Transfection, and Infection

The lentiviral clone expressing short hairpin (sh) RNA targeting-LDOC1 (shLDOC1)-, pLKO.1-shLDOC1-puro, is constructed using the LDOC1 knockdown plasmid (TRCN0000118179 containing DNA sequence encoding shLDOC1, 5′-CCGGGCTCGTGAACGAGAACCGATTCTCGAGAATCGGTTCTCGTTCACGAGCTTTT TG-3′ (SEQ ID No. 2; i.e., a DNA sequence encodes siRNA targeting LDOC1)) and the pLKO.1 vector and plasmid TRCN0000118179 are provided by RNA Technology Platform and Gene Manipulation Core Facility, Academia Sinica, Taipei, Taiwan. Through LDOC1 knockdown using lentivirus, stable cell lines are established from PC9 and HCC827 and are named PC9-shLDOC1 and HCC827-shLDOC1, respectively, as per the procedure of RNAi core. PC9-shLDOC1, HCC827-shLDOC1, and the corresponding control (shCtrl) cells are selected with puromycin (5 μg/mL). The western blot analysis of whole cell lysates is performed using the customized anti-LDOC1 antibodies (Abclonal, Woburn, MA, USA), which confirmed that shCtrl cells produced LDOC1 proteins at amounts similar to those detected in parental cells, and that shLDOC1 cells produced approximately 70% less LDOC1 protein relative to shCtrl cells. Plasmid-expressing FLAG-tagged human AP1M1 and AP2M1 are purchased from OriGene (MD, USA), and plasmid-expressing V5-tagged human LDOC1 is generated using a pcDNA6.2-DEST mammalian expression vector (Thermo Fisher). All these plasmids are validated for correct ORF through DNA sequencing. U2OS cells are subcultured 16 h before transfection with lipofectamine 2000 (Invitrogen, Carlsbad, CA, USA), as per the manufacturer's protocol.

### Western Blot Analysis and Antibodies

Western blot analysis is performed as per the standard procedure. In brief, total protein is harvested using cell lysis buffer (Thermo Fisher) containing a protease inhibitor cocktail and phosphatase inhibitors (Thermo Fisher). The protein concentration of cellular lysates is determined using a Pierce BCA Protein Assay Kit (Thermo Fisher). Proteins are separated through SDS-PAGE and are electroblotted onto PVDF membranes (Merck Millipore, Danvers, MA, USA). The membranes are blocked for 1 h at ambient temperature with 5% skimmed milk or BSA and subsequently incubated overnight at 4° C. with primary antibodies. On the next day, the membranes are washed with Tris-buffered saline with 0.1% Tween® 20 detergent (TBST) and incubated with horseradish peroxidase- (HRP-) conjugated goat anti-rabbit IgG (GTX213110-01) for 1 h at room temperature. The blots are developed using ClarityWestern ECL Substrate (BioRad, Hercules, CA, USA). HRP-conjugated EasyBlot Anti-Rabbit IgG (GTX #221666, GeneTex, Irvine, CA, USA) is used as the secondary antibody for immunoprecipitation samples. To detect LDOC1, the mPAGE Bis-Tris SDS-PAGE Precast Gel system (Millipore) is used according to the manufacturer's instruction. All antibodies used in Western blot analysis can be purchased from commercial companies, except for custom polyclonal anti-LDOC1 antibodies, which are produced by ABclonal. The custom polyclonal anti-pAXLY779 (anti-phospho-AXLY779) antibodies are gifts from Dr. Chuang, S. E. (National Health Research Institutes). The antibodies used in the present disclosure are anti-EGFR (GTX121919), anti-pEGFRY1068 (GTX132810), anti-HER2 (GTX100509), HER3 (GTX100256), anti-GAPDH (GTX100118), anti-AP1M1 (GTX64907), and anti-AP2M1 (GTX #113332), which are purchased from GeneTex; anti-AXL (C89E7), which is purchased from Cell Signaling Technology; anti-pHER2Y1248 (AF1768-SP) and anti-pHER3Y1262 (anti-phospho-HER3Y1262) (AF5817-SP), which are purchased from R&D system; and anti-EGFR (51071-2-AP, Protech, Rosemont, IL, USA), which is purchased from Protech.

### Co-Immunoprecipitation

The cells are serum-starved overnight and then stimulated with EGF (10 mM) for 30 min before protein lysates are harvested for a co-immunoprecipitation assay. Subsequently, the cells are washed with PBS and lysed in Pierce IP Lysis Buffer (Thermo Fisher) containing a protease inhibitor cocktail and phosphatase inhibitors (Thermo Fisher). After the cells are centrifuged at 13,000×g and 4° C. for 10 min, the protein concentration of the supernatant is measured, and 700 μg to 1000 μg of protein lysates are incubated with the indicated antibodies at 4° C. overnight and subsequently with protein A-magnetic beads (BioTools, Dalian, China) for 1 h at room temperature. After the immunoprecipitants are thoroughly washed, the immunoprecipitants are eluted and subjected to Western blot analysis, as per the standard procedure. The antibodies used in the co-immunoprecipitation experiments are custom anti-LDOC1 (ABclonal), anti-AP1M1 (12112-1-AP, Protech), anti-AP2M1 (144-02492, RayBiotech, Norcross, GA, USA), and anti-EGFR (51071-2-AP, Protech).

### Double Immunofluorescence Staining

For the analysis of the colocalization of ectopically expressed V5-tagged LDOC1 with FLAG-tagged AP1M1 or AP2M1, U2OS cells are seeded onto coverslips and allowed to adhere overnight before DNA transfection. One day after the cells underwent transfection, the cells are starved overnight with medium containing 0.05% FBS. After the cells are treated with EGF (10 nM for 30 min), the cells are fixed with methanol at −20° C. for 10 min. Coverslips are then washed in PBS and incubated in blocking solution (5% normal goat serum in PBS) for 30 min at room temperature. The cells are then subjected to double immunofluorescence staining with the primary antibodies of rabbit pAb to V5-tag (14440-1-AP, Protech) and mouse mAb to FLAG-tag DYKDDDDK (SEQ ID NO. 3) (66008-4-Ig, Protech), and the secondary antibodies of CoraLite594-conjugated goat antirabbit IgG (SA00013-4, Protech) and CoraLite488-conjugated goat anti-mouse IgG (SA00013-1, Protech). For the analysis of the association of EGFR with adaptin AP1M1 or AP2M1, A549 or PC9 cells are seeded onto coverslips and allowed to adhere overnight before they are starved overnight with medium containing 0.05% FBS. Next, fixation and blocking are performed as per the aforementioned procedures, after which double immunofluorescence staining was performed using mouse mAb to EGFR (66455-1-Ig, Protech) and rabbit pAb to AP1M1 (12112-1-AP, Protech) or AP2M1 (144-02492, RayBiotech) as primary and secondary antibodies, respectively, as per the aforementioned procedure. The immunostained cells are washed thoroughly with PBS, and the nuclei of the cells are counterstained with DAPI (100 μg/mL, Sigma-Aldrich, St. Louis, MO, USA). The stained cells are then incubated in buffer containing 0.1 M PBS, pH 8.0; 2% n-propyl gallate; and 60% glycerol, and the obtained slides are analyzed using a Leica TCS SP5 II confocal microscope (Wetzlar, Germany) and Leica LAS AF software (version 4.0).

### Endocytosis Assay for EGFR Internalization and Recycling

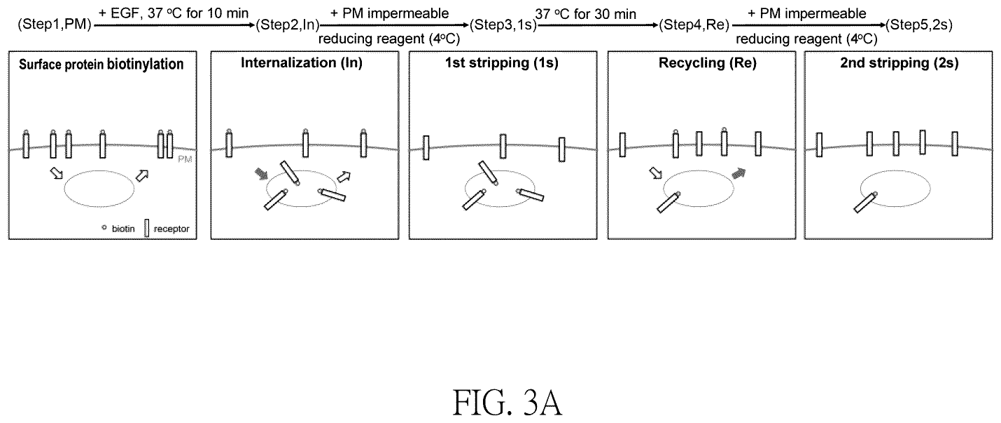

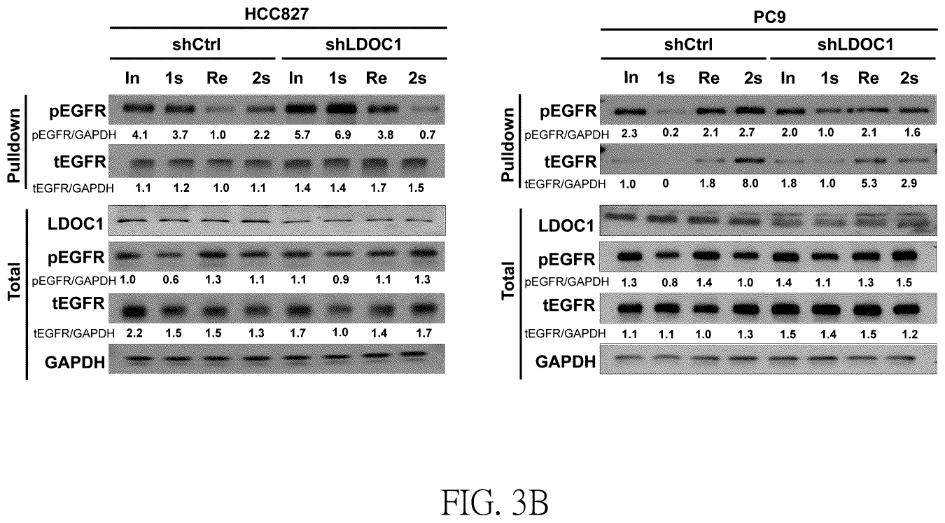

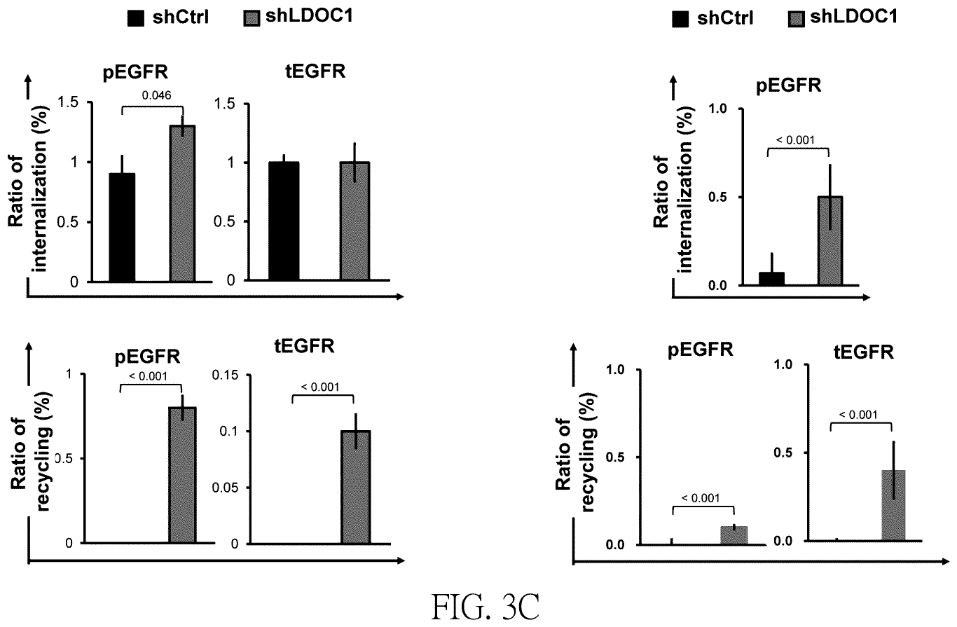

Endocytosis assay is performed using a Pierce cell surface protein biotinylation and isolation kit (Thermo Fisher), as per a modified version of the method described by Uemura (Uemura, T.; Suzuki, T.; Dohmae, N.; Waguri, S. Clathrin adapters AP-1 and GGA2 support expression of epidermal growth factor receptor for cell growth. Oncogenesis 2021, 10, 80). The principle of EGFR endocytosis assay is shown in FIG. 3A. In brief, the cells are serum-starved overnight. After the cells are briefly washed with ice-cold PBS, they are incubated with 0.5 mM EZ-Link Sulfo-NHS-SS-biotin (Thermo Fisher) in ice-cold PBS for 30 min. After the cells are washed twice with ice-cold PBS, the cells are incubated in DMEM for 30 min at 37° C. or in DMEM containing 10 nM EGF for 10 min at 37° C. to induce the internalization of biotinylated EGFR (Steps 1 to 2, PM to In). Subsequently, the cells are incubated with a membrane-impermeable stripping solution (50 mM glutathione, 75 mM NaCl, 75 mM NaOH, 1% BSA, and 10 mM EDTA) on ice for 15 min to remove the remaining biotin on the surfaces of the cells (Step 3, 1s). The cells are re-incubated in DMEM at 37° C. for 30 min for the recycling of internalized EGFR (Step 4, Re), after which cells undergo a second treatment with or without the stripping solution (Step 5, 2s). Cellular protein lysates are harvested immediately after In, 1s, Re, and 2s. The lysates are treated with avidinconjugated agarose for pulldown, and the recovered proteins are analyzed using Western blotting. The protein lysates are harvested immediately after biotinylation and are subjected to pulldown using avidin-conjugated agarose, then the recovered proteins are used as a PM fraction for analysis. The internalization ratio of EGFR is calculated by dividing the amount of biotinylated EGFR detected after the first stripping by that detected in the absence of the stripping reagent (1s/In). The biotinylated EGFR (pulldown) detected in Step 4 (Re) is equal to the sum of the EGFR recycled to the PM and that retained within the cells (internalized and un-recycled EGFR). Thus, to obtain the ratio of recycled EGFR to internalized EGFR, the amount of biotinylated EGFR detected after the second stripping is subtracted from that detected in the absence of the stripping reagent, and the obtained value is further divided by that detected in the absence of the stripping agent (i.e., (Re−2s)/Re). The assay is verified to be performed within a range where the band intensity is linearly proportional to the protein concentration.

### Immunohistochemistry (IHC) Staining and Hematoxylin and Eosin (HE) Staining

Formalin-fixed, paraffin-embedded tissue blocks are sliced into 5-μm sections for IHC and HE staining. In brief, slides are dipped in hematoxylin solution for 5 min; washed in distilled water; dipped in eosin solution for 2 min; and routinely processed and stained with either a customized anti-LDOC1 (ABclonal) or an anti-EGFR (EP22, Zeta, New York, NY, USA), biotinylated immunoglobulins, and a super-sensitive HRP label system as per the protocol described previously. Slides with tissue sections (thickness of 5 μm) are deparaffinized with xylene and rehydrated using graded alcohols. Heat-induced antigen retrieval is performed using a citric acid buffer (pH of 6.0) for 30 min. After endogenous peroxidase activity is blocked with 0.5% H₂O₂ in methanol for 30 min, the sections are incubated with a customized anti-human LDOC1 antibody (1:150; Abclonal) or an anti-human EGFR (1:100; EP22, Zeta) at 4° C. overnight. After washing is completed, the specific signals on each section are developed using a BOND-PRIME Polymer DAB chromogen. LDOC1 immunoreactivity is classified as either negative (absent or weak cytoplasmic staining) or positive (moderate or strong cytoplasmic staining). EGFR immunoreactivity is evaluated and classified as membranous or cytoplasmic staining on the basis of the predominant locations of immunoreactivity. HE staining is conducted as per the manufacturer's protocol.

### MTT Assay for EGFR-TKI Sensitivity

Cells are seeded at a density of 5×10³ per well in a 96-well plate. The next day, the cells are treated with the indicated concentrations of gefitinib, erlotinib, and sunitinib. An appropriate amount of DMSO is also added to the control cells. After 72 h of treatment, the cells are incubated with a solution of MTT (3-(4,5-dimethylthiazol-2-yl)-2,5-diphenyltetrazolium bromide) (Sigma-Aldrich) for 4 h at 37° C. Subsequently, the medium is aspirated, and 100 μL DMSO is added to solubilize the formazan. Colored formazan converted from MTT using viable cells is measured at 570 nm with a microplate reader (BioRad). Experiments are performed in triplicate. IC50 values are determined using the scientific and statistical software GraphPad Prism 10.0.0.

### Colony Formation Assay for Gefitinib Sensitivity

The cells are seeded at a density of 3×10² per 35-mm dish and are allowed to adhere overnight before gefitinib treatment. The cells are then incubated in medium containing varying concentrations of gefitinib at 37° C. for 10 days. Gefitinib is dissolved in DMSO, and the cells grown in medium containing 0.002% DMSO (solvent control). Colonies are counted after Giemsa staining, as per the protocol described in previous study (Lee, C. H.; Yang, J. R.; Chen, C. Y.; Tsai, M. H.; Hung, P. F.; Chen, S. J.; Chiang, S. L.; Chang, H.; Lin, P. Novel STAT3 Inhibitor LDOC1 Targets Phospho-JAK2 for Degradation by Interacting with LNX1 and Regulates the Aggressiveness of Lung Cancer. Cancers 2019, 11, 63).

### Statistical Analysis

In the present disclosure, statistical analyses are conducted using the statistical software PASW Statistics (version 18.0) (IBM Corporation, Armonk, NY, USA). The clinicopathological characteristics of patients with advanced NSCLC and the associations between LDOC1 expression and various clinicopathological factors are evaluated through an X2 test or Fisher's exact test. The influence of clinicopathological characteristics on overall survival was analyzed using a Cox proportional hazards model. The p values obtained through the Wald test are recorded. Kaplan-Meier survival analysis with a log-rank significance test is performed to estimate the probability of survival. A p value of less than 0.05 was regarded as statistically significant.

### Results

### LDOC1 Bound to Subunits of Clathrin Adaptor Complexes in NSCLC Cells

On the basis of the predicted interaction between LDOC1 and AP1M1 in the STRING database and the presence of multiple binding motifs for clathrin adaptors in LDOC1 (FIG. 2A), it is speculated that LDOC1 is involved in CME of EGFR by interacting with subunits of clathrin adaptor proteins, AP1M1 and AP2M1. To enable investigation of the associations between LDOC1 and clathrin adaptor proteins, cells are serum-starved overnight and then treated with EGF (10 nM) for 30 min before being analyzed using co-immunoprecipitation (coIP)-western blot (WB) assays. The results of this analysis support interactions of LDOC1 with AP1M1 and AP2M1 in human NSCLC cell lines, EGFRWT A549, and EGFRM PC9 cells (FIG. 2B and FIG. 2C). Notably, an abundance of LDOC1-associated AP2M1 is observed in PC9 cells. To confirm these interactions, double immunofluorescence staining with confocal microscopy analysis is performed. U2OS cells are co-transfected with plasmids expressing V5-tagged LDOC1 and FLAG-tagged AP1M1 or AP2M1. After transfection, the cells are serum-starved for 6 h before EGF treatment for 30 min. Immunostaining of exogenous V5-LDOC1 (red) and FLAG-AP1M1 (green) or AP2M1 (green) revealed widespread distribution of LDOC1 throughout the transfectants, with strong staining detected in numerous punctate structures around the nucleus and weak staining inside the nucleus (FIG. 2D and FIG. 2E). AP1M1 and AP2M1 are predominantly localized to cytoplasmic punctate structures and colocalized with LDOC1 (FIG. 2D and FIG. 2E; white speckles). Additionally, several adaptins are irregularly distributed on the plasma membrane and also colocalized with LDOC1 (FIG. 2D and FIG. 2E; white dots). Altogether, these results reveal associations between LDOC1 and subunits of clathrin adaptors in NSCLC cells.

### LDOC1 Depletion Supports Either Internalization or Recycling of EGFR in EGFR^{M} NSCLC Cells

Given the importance of clathrin adaptors in CME, it is suggested that LDOC1 regulates CME of EGFR through competition with the receptor to bind to clathrin adaptors. To test this suggestion, the effect of LDOC1 depletion on the internalization and recycling of EGFR in two EGFR^{M} NSCLC cell lines, PC9 and HCC827 is measured, by using previously described cell surface labeling methods (FIG. 3A). Cells are serum-starved overnight prior to EGF (10 nM) treatment. In the HCC827 cells, LDOC1 depletion slightly but significantly increased the internalization of phosphorylated EGFR (pEGFR), with the internalization ratio increasing from 0.9 to 1.3; however, LDOC1 depletion has no effect on the internalization of total EGFR (tEGFR). PM recycling of pEGFR and tEGFR is dramatically enhanced in the LDOC1-depleted HCC827 cells but completely abolished in the LDOC1-expressing control cells (FIG. 3B). Under the same conditions, internalization of pEGFR in the LDOC1-depleted PC9 cells increased fivefold compared with internalization in the control cells, with the internalization ratio increasing from <0.1 to 0.5. Total EGFR is detected in the intracellular portion (2s) of the control cells. A trace amount of tEGFR was recycled to the PM (Re-2s) in the LDOC1-depleted PC9 cells (FIG. 3C). Thus, as was the case with the HCC827 cells, LDOC1 depletion facilitated the recycling of pEGFR and tEGFR to the PM, and receptor recycling is completely inhibited in the control cells because the value of (Re-2s) is negative (FIG. 3C). In total protein lysates (total), the expression of pEGFR and tEGFR in the shLDOC1 and shCtrl HCC827 cells is comparable; however, the expression of pEGFR and tEGFR in the PC9-shLDOC1 cells is higher than that in the PC9-shCtrl cells. These results, together with the aforementioned coIP-WB and colocalization data (FIG. 2B to FIG. 2E), suggest that LDOC1 depletion promotes either the CMI or recycling of EGFR through CME in EGFR^{M} NSCLC cells.

### Depletion of LDOC1 Leads to Upregulation of the Potential EGFR-Interacting RTKs

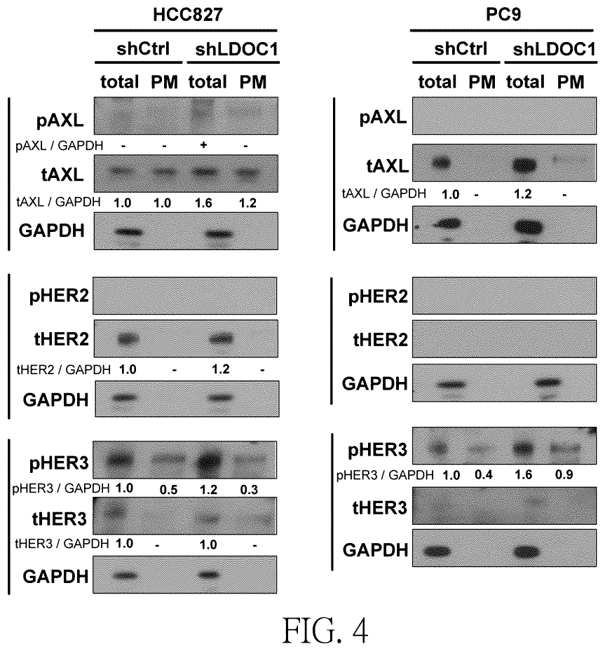

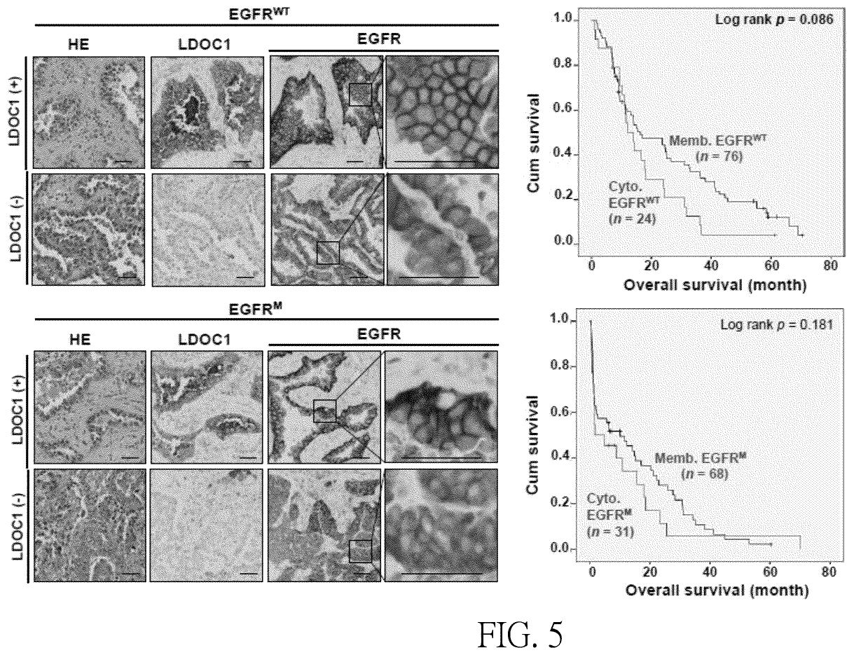

Whether the cellular transport of the potential EGFR-interacting RTKs may be regulated by LDOC1 is explored. Both total and PM levels of phosphorylated and total AXL, HER2, and HER3 in HCC827 and PC9 cell lines is evaluated by using the surface protein biotinylation method as described above (FIG. 3A). Using this method of the present disclosure, phosphorylated HER2 and AXL are not detected for both total and PM lysate fractions of PC9 and HCC827 cells (FIG. 4). In contrast, pHER3 is primarily present in the total lysates of the two cell lines, and the levels in shLDOC1 cells are more abundant than those in control cells (FIG. 4). It is suggested that HCC827 and PC9 cells express cytoplasmic pHER3, independent of LDOC1 expression. Total AXL (tAXL) is observed in total and PM lysate preparations of HCC827 and total lysates of PC9 cells, and slightly increased in shLDOC1 PC9 and HCC827 cells. Similar results are obtained for total HER2 (tHER2) and HER3 (tHER3) (FIG. 4).

LDOC1 Downregulation Associated with Cytoplasmic EGFR Expression in NSCLC Tumors

The efficiency of the internalization and recycling processes is a key factor affecting the subcellular distribution of proteins. To examine whether LDOC1 affects the subcellular distribution of EGFR in NSCLC, immunohistochemical analyses of 100 EGFR^{WT} and 100 EGFR^{M} NSCLC tumors are performed. The clinicopathological characteristics of patients with EGFR^{WT} and EGFR^{M} advanced NSCLC stratified by LDOC1 level are presented in Tables 1 and 2, respectively. Low LDOC1 expression is observed in 45% (45/100) and 42% (42/100) of patients with EGFRWT and EGFRM, respectively. In EGFRWT NSCLC, cytoplasmic EGFR is observed in 9.1% and 42.2% of patients with high and low LDOC1 expression, respectively. In EGFR^{M} NSCLC, cytoplasmic EGFR is observed in 12.1% and 59.5% of patients with high and low LDOC1 expression, respectively. Membranous EGFR is noticeably reduced and barely detectable in LDOC1-depleted EGFR^{WT} and EGFR^{M} NSCLC cases. Although no associations are observed between LDOC1 expression and clinicopathological characteristics, including age, sex, and cancer stage, LDOC1 depletion is significantly associated with cytoplasmic EGFR expression (p<0.001) in most patients. Representative immunohistochemical images for cytoplasmic EGFR in EGFR^{WT} and EGFR^{M} NSCLC tumors are presented in upper left panel of FIG. 5 and lower left panel of FIG. 5, respectively. Whether cytoplasmic EGFR is associated with NSCLC prognosis is then investigated. As shown in upper right panel of FIG. 5 and lower right panel of FIG. 5, the survival rate for patients with tumor-expressing membranous EGFR is not significantly higher than those who are expressing cytoplasmic EGFR in EGFR^{WT} and EGFR^{M} cases (p=0.086 and 0.181, respectively). Collectively, the results indicated that LDOC1 downregulation is associated with cytoplasmic EGFR in EGFR^{WT} and EGFR^{M} NSCLC, possibly as a consequence of accelerated internalization.

TABLE 1
Clinicopathological characteristics of
patients with EGFR^{WT} advanced NSCLC.

| | | No. (%) | | |
|---|---|---|---|---|
| | | LDOC1 High | LDOC1 Low | |
| | | Expression | Expression | |
| Characteristics | No. | n = 55 | n = 45 | p Value |
| Age | | | | 0.933 |
| ≤75 years | 36 | 20 (36.4) | 16 (35.6) | |
| >75 years | 64 | 35 (63.6) | 29 (64.4) | |
| Gender | | | | 0.841 |
| Male | 50 | 27 (49.1) | 23 (51.1) | |
| Female | 50 | 28 (50.9) | 22 (48.9) | |
| Stage | | | | 0.888 |
| IIIB + IIIC | 15 | 8 (14.5) | 7 (15.6) | |
| IVA + IVB | 85 | 47 (85.5) | 38 (84.4) | |
| EGFR expression | | | | <0.001 |
| Membranous | 76 | 50 (90.9) | 26 (57.8) | |
| Cytoplasmic | 24 | 5 (9.1) | 19 (42.2) | |

TABLE 2
Clinicopathological characteristics of
patients with EGFR^{M} advanced NSCLC.

| | | No. (%) | | |
|---|---|---|---|---|
| | | LDOC1 | | |
| | | High | LDOC1 Low | |
| | | Expression | Expression | |
| Characteristics | No. | n = 58 | n = 42 | p Value |
| Age | | | | 0.276 |
| ≤75 years | 54 | 34 (58.6) | 20 (47.6) | |
| >75 years | 46 | 24 (41.4) | 22 (52.4) | |
| Gender | | | | 0.714 |
| Male | 45 | 27 (46.6) | 18 (42.9) | |
| Female | 55 | 31 (53.4) | 24 (57.1) | |
| Stage | | | | 0.177 |
| IIIB + IIIC | 18 | 13 (22.4) | 5 (11.9) | |
| IVA + IVB | 82 | 45 (77.6) | 37 (88.1) | |
| EGFR expression* | | | | <0.001 |
| Membranous | 68 | 51 (87.9) | 17 (40.5) | |
| Cytoplasmic | 31 | 7 (12.1) | 24 (59.5) | |

*indicates that one case is not assessed.

### LDOC1 Depletion Enhances and Sustains Prolonged Activation of EGFR, AXL, and HER2 in EGFR^{M} Cells

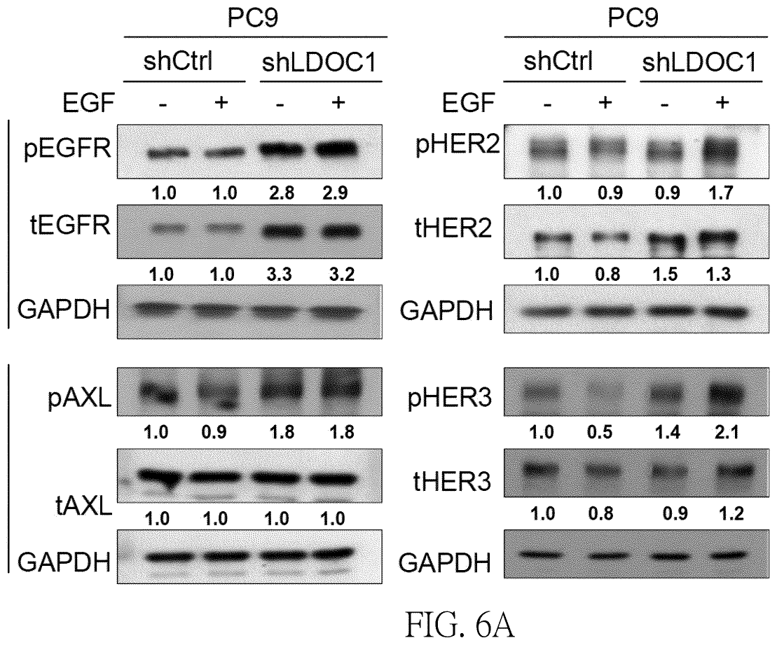

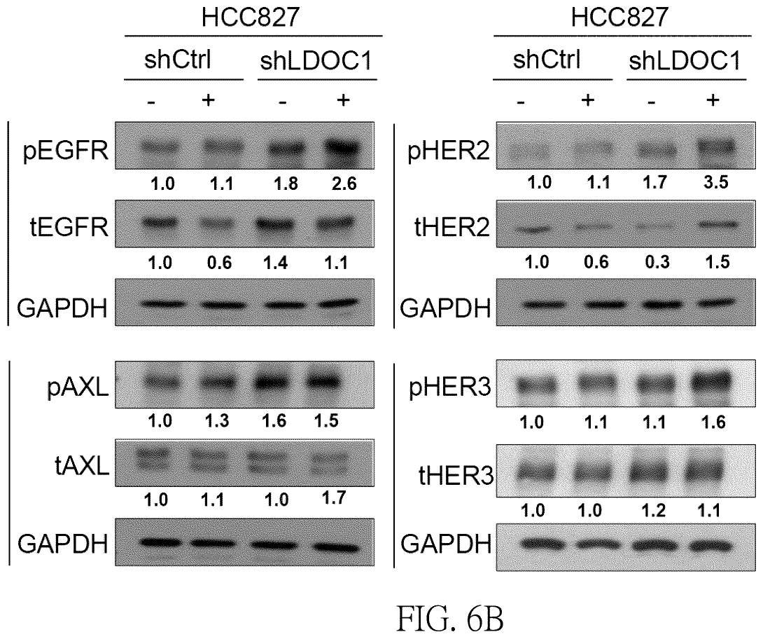

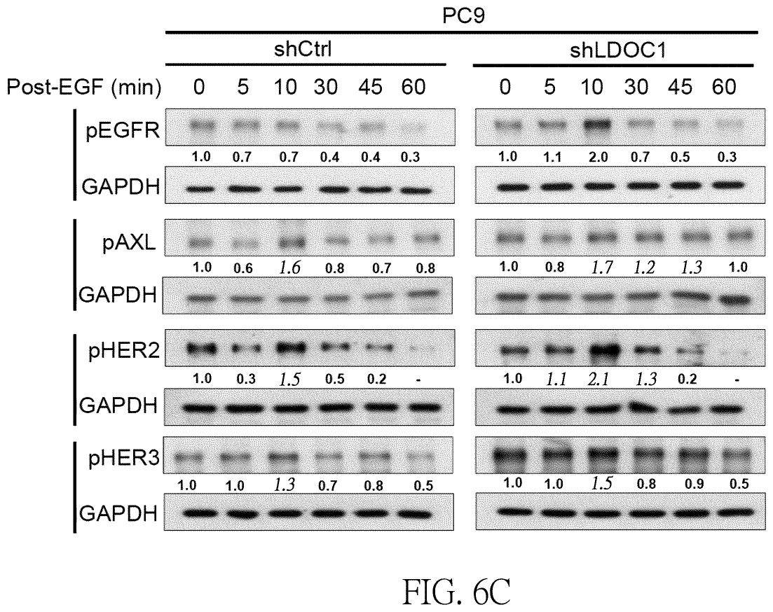

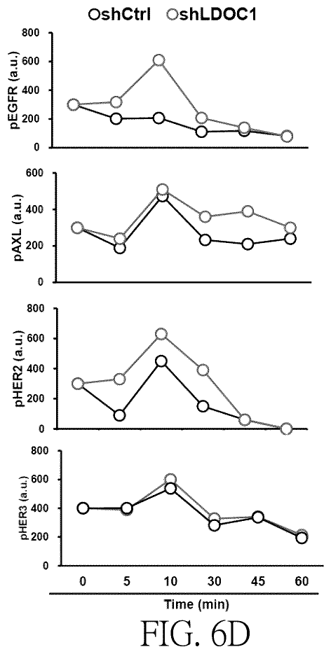

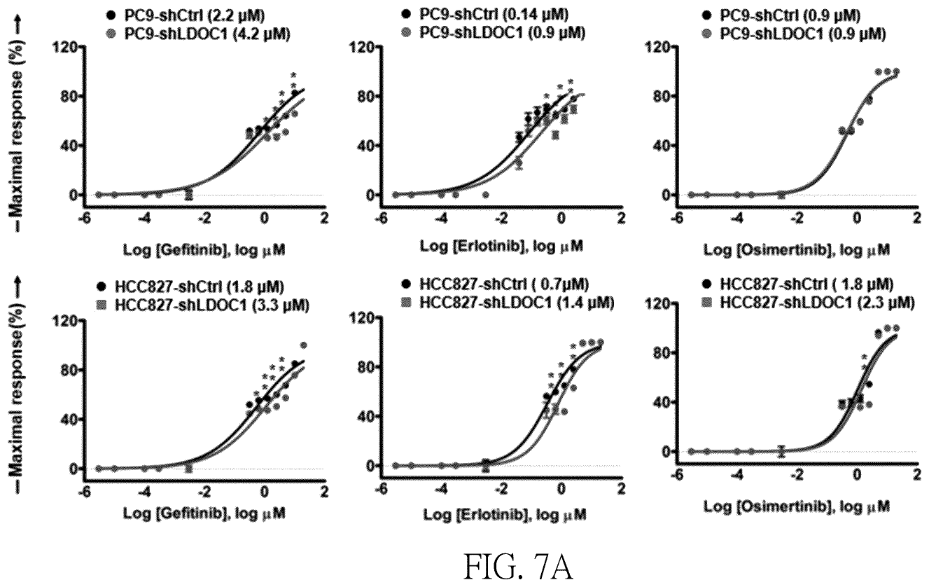

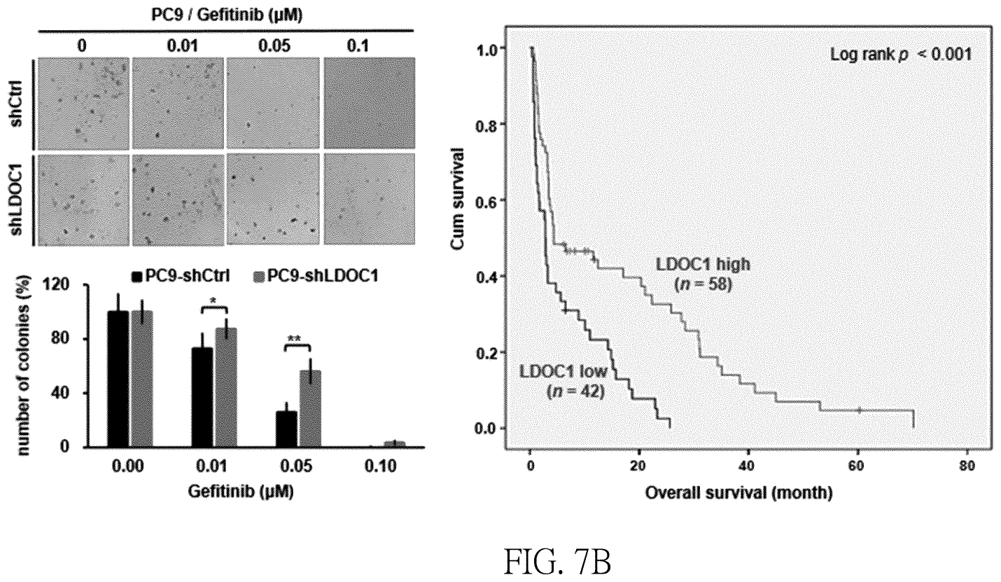

LDOC1 may regulate the endocytosis of EGFR through CME is demonstrated (FIG. 2A-FIG. 2E; and FIG. 3A-FIG. 3C). Although CME has been shown to either attenuate or enhance RTK signaling, whether it modulates EGFR signaling in NSCLC remains unclear. Thus, the effect of LDOC1 depletion on the activation of EGFR and its related RTKs, including HER2, HER3, and AXL in EGFRM NSCLC cells is investigated. In the PC9 cells, LDOC1 depletion approximately doubled pEGFR, pAXL, and pHER2 expression, regardless of EGF stimulation. LDOC1 depletion obviously increased the levels of pHER3 upon EGF stimulation, and tEGFR and tHER2 expression is higher in the LDOC1-depleted PC9 cells than in the control cells, regardless of EGF stimulation (FIG. 6A). Similar results are obtained from HCC827 cells, but the magnitude of the increase in pEGFR, pAXL, pHER2, and pHER3 caused by LDOC1 depletion is not as dramatic as in PC9 cells under unstimulated conditions (FIG. 6B). These results indicate that LDOC1 depletion enforces the activation of EGFR, HER2, HER3, and AXL in EGFR^{M} NSCLC cells. If depletion of LDOC1 supports prolonged activation of EGFR, HER2, HER3, and AXL is then further examined. To test this, Changes in the expression of phosphorylated EGFR, HER2, HER3, and AXL in shCtrl and shLDOC1 PC9 cells after stimulation with EGF (10 nM) for 10 min (FIG. 6C and FIG. 6D) is monitored. The pEGFR expression in the LDOC1-expressing control cells does not further increase with EGF stimulation. However, the levels of pAXL and pHER2 in the LDOC1-expressing control cells increased, peaking at 10 min, and then sharply declined. Conversely, after EGF stimulation, expression of pEGFR in the LDOC1-depleted PC9 cells increased, peaking at 10 min, and then declined, and expression of pAXL and pHER2 increased, peaking at 10 min, but only starting to decline after 30 and 45 min, respectively. LDOC1 depletion does enhance the abundance of pHER3 but has no effect on prolonged HER3 activation. Altogether, these results demonstrate that depletion of LDOC1 not only activates EGFR, HER2, and AXL but also prolongs these RTKs' signaling.

LDOC1 Downregulation Reduces Sensitivity to First-Generation EGFR-TKIs and Predicts Worse Outcomes in Patients with EGFR^{M} NSCLC Who are Treated with Gefitinib

First-generation EGFR-TKIs, such as gefitinib and erlotinib, are the standard first-line treatments for patients with EGFR^{M} NSCLC. Given that LDOC1 depletion enhances and prolongs activation of EGFR, AXL, HER2, and HER3 (FIG. 6A to FIG. 6D), it is believe that LDOC1 depletion affects EGFR-TKI sensitivity in EGFR^{M} NSCLC cells. The effects of gefitinib, erlotinib, and osimertinib (a third-generation EGFR-TKI) on the viability of shLDOC1 and shCtrl PC9 and HCC827 cells by using MTT assays are compared. After treatment for 72 h, the half-maximal inhibitory concentration (IC50) values of gefitinib and erlotinib are significantly higher in the shLDOC1 cells than in the shCtrl cells, and the IC50 values of osimertinib are the same in both the shLDOC1 and shCtrl PC9 cells (0.9 μM; upper panel of FIG. 7A) and increased from 1.8 (shCtrl) to 2.3 (shLDOC1) μM in HCC827 cells (lower panel of FIG. 7A). A colony-forming assay is performed to confirm the reduction in gefitinib sensitivity in the PC9 cells caused by LDOC1 depletion. Consistently, LDOC1 depletion increased the number of cell colonies in the gefitinib-treated PC9 cells (left panel of FIG. 7B). Reduced sensitivity to gefitinib may lead to poorer overall survival in patients with EGFR^{M} advanced NSCLC receiving gefitinib treatment. To test this, the association between LDOC1 expression and prognosis in patients with EGFR^{M} advanced NSCLC who received gefitinib is assessed. Kaplan-Meier analysis reveals that LDOC1 downregulation is strongly associated with shorter overall survival (p<0.001; right panel of FIG. 7B). The results of univariate analysis indicated that the risk factors for shorter overall survival included older age (>75 years), late stage (IVA or IVB), and low LDOC1 levels (p=0.003, <0.001, and <0.001, respectively), and all of these risk factors remained significant in multivariate analysis (p=0.014, 0.009, and 0.002, respectively; Table 3). Notably, low LDOC1 (p=0.002) is more strongly associated with shorter overall survival than late stage (p=0.009). Taken together, results of the present disclosure show that LDOC1 depletion considerably decreased gefitinib and erlotinib sensitivity in PC9 cells. Furthermore, LDOC1 downregulation is an independent factor associated with poor overall survival in gefitinib-treated patients with EGFR^{M} advanced NSCLC.

TABLE 3
Univariate and multivariate analyses of overall survival of
patients with EGFR^{M} advanced NSCLC who received gefitinib.

| | | Univariate Analysis | | Multivariate Analysis | |
|---|---|---|---|---|---|
| | | Overall Survival | | Overall Survival | |
| Covariate | No. | HR^{a} (95% C.I.^{b}) | p Value | HR (95% C.I.) | p Value |
| Age | | | 0.003 | | 0.014 |
| ≤75 years | 54 | 1 | | 1 | |
| >75 years | 46 | 1.92 (1.24-2.96) | | 1.73 (1.12-2.67) | |
| Gender | | | 0.697 | | |
| Male | 45 | 1 | | | |
| Female | 55 | 1.09 (0.72-1.65) | | | |
| Stage | | | <0.001 | | 0.009 |
| IIIB + IIIC | 18 | 1 | | 1 | |
| IVA + IVB | 82 | 3.46 (1.63-7.35) | | 2.80 (1.29-6.05) | |
| LDOC1 | | | <0.001 | | 0.002 |
| expression | | | | | |
| High | 58 | 1 | | 1 | |
| Low | 42 | 2.36 (1.50-3.70) | | 2.02 (1.28-3.19) | |
| EGFR | | | 0.181 | | |
| expression^{c} | | | | | |
| Membranous | 68 | 1 | | | |
| Cytoplasmic | 31 | 1.36 (0.87-2.15) | | | |

^{a}indicates Hazard ratio; ^{b}indicates confidence interval; ^{c}indicates that one case is not assessed; and C.I. indicates confidence intervals.

### Discussion

Although gefitinib and erlotinib is the first-line treatment for patients with NSCLC harboring EGFR^{M} in many countries, 30% to 50% of patients with EGFR^{M} do not benefit from it. The molecular features of gefitinib-sensitive tumors must be identified, and novel therapeutic targets associated with gefitinib-resistance should be developed. The present disclosure demonstrates that LDOC1 is a critical factor affecting EGFR-TKI (e.g., gefitinib) efficacy (right panel of FIG. 7B: Table 3). Because LDOC1 possesses multiple binding motifs for clathrin adaptors (FIG. 2A to FIG. 2E), it may interfere with the formation of the EGFR-AP1 and EGFR-AP2 complexes by binding to AP1M1 and AP2M1. Therefore, depletion of LDOC1 may facilitate the internalization and recycling of EGFR through CME (FIG. 3B).

It is found that LDOC1 depletion enhances EGFR activation (FIG. 6A and FIG. 6B) because CMI of EGFR is not required for receptor degradation but is essential for maintaining prolonged EGFR signaling. Moreover, depletion of LDOC1 not only enhanced EGFR signaling but also augmented the activation of HER2, HER3, and AXL (FIG. 6A to FIG. 6D). In agreement with the importance of AXL, HER2, and HER3 to EGFR-TKI sensitivity in NSCLC, LDOC1 depletion reduced EGFR-TKI sensitivity in PC9 and HCC827 cells (FIG. 7A). This reduced sensitivity may be why patients with LDOC1 depletion have worse prognoses than those without LDOC1 depletion (Left panel of FIG. 7B). Augmented activation of AXL may play a critical role in gefitinib and erlotinib resistance in LDOC1-depleted PC9 and HCC827 cells because LDOC1 downregulation causes pAXL to significantly increase, regardless of the presence of EGF (FIG. 6A and FIG. 6B).

Osimertinib is a third-generation, irreversible EGFR-TKI that selectively inhibits both EGFR-TKI-sensitizing and EGFR-T790M-resistant mutations. Notably, osimertinib has almost the same inhibitory effect on the proliferation of shCtrl and shLDOC1 PC9 cells. Upper panel of FIG. 7A displays the finding that supports the assumption that mutagenesis driven by AXL may lead to the emergence of uncommon mutations of EGFR, such as T790M in shLDOC1 EGFR^{M} NSCLC cells, and confers resistance to gefitinib and erlotinib in LDOC1-depleted PC9 cells. In the present application, it is demonstrated that LDOC1-depleted PC9 cells survive and form colonies after long-term (10-day) treatment with gefitinib.

By contrast, no colonies of PC9-shCtrl cells are observed under the same conditions (Left panel of FIG. 7B). These results suggest LDOC1 depletion confers acquired resistance to PC9 cells.

The present disclosure provides a promising treatment strategy for LDOC1 (−) EGFR^{M} NSCLC by using EGFR-TKIs in combination with an AXL inhibitor. In addition, because the EGFR-AXL complex has been detected in brain tumor cells, and EGFR can form heterodimers with HER2 and HER3, LDOC1 may also affect cellular transport, including internalization and PM recycling of these EGFR-interacting partners. It is discovered that LDOC1 depletion increased the internalization of AXL, suggesting that interactions between EGFR and AXL may also exist in NSCLC cells. In addition to increasing the levels of EGFR, AXL, HER2, and HER3, upregulation of cytoplasmic EGFR and reduced membranous EGFR (lower left panel of FIG. 5 and Table 2) may contribute to reducing the efficacy of gefitinib in patients with LDOC1 (−) EGFR^{M} NSCLC, given that the cytoplasmic location of EGFR can hinder drug targeting. In PC9 and HCC827 cells, LDOC1 depletion facilitates both the internalization and recycling of EGFR, and LDOC1 downregulation results in a reduction in membranous EGFR.

Osimertinib (rather than gefitinib or erlotinib), anti-AXL monoclonal antibodies, and targeted autophagy may improve the efficacy of EGFR-targeting therapy for LDOC1 (−) EGFR^{M} NSCLC. Our observations may also reveal a novel and potent strategy for overcoming LDOC1 downregulation-induced activation of members of the ErbB family and AXL. Adeno-associated viral vectors are useful in gene therapy. Clinical testing should be considered for adeno-associated viruses carrying an LDOC1 open reading frame. In summary, the present disclosure not only highlights the importance of targeting EGFR trafficking in medical oncology but also reveals a novel mechanism involving tumor suppressor genes.

The advantages of the present disclosure are provided below. The prediction accuracy for the efficacy of EGFR-TKI (e.g., gefitinib) is significantly improved. As a result, patients who do not respond to EGFR-TKI (e.g., gefitinib) may promptly adopt alternative treatment strategies, increasing their chances of success. Additionally, the burden on healthcare can be reduced due to decreased usage of EGFR-TKI (e.g., gefitinib). It is a win-win invention for both patients and medical units. Moreover, the present disclosure also offers the advantages of low manufacturing costs and a low implementation technical threshold. For example, IHC test offers the advantages of low executing costs and a low implementation technical threshold, since IHC test is widely used in medical research laboratories as well as clinical diagnostics to predict therapeutic response.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A method for predicting drug efficacy of lung cancer, comprising:
providing a biological sample of a subject with lung cancer;
analyzing an expression level of Leucine Zipper Down-regulated in Cancer 1; and
predicting the drug efficacy based on the expression level of the Leucine Zipper Down-regulated in Cancer 1.

2. The method of claim 1, wherein the drug efficacy is an efficacy of an epidermal growth factor receptor-tyrosine kinase inhibitor on the subject.

3. The method of claim 2, wherein the epidermal growth factor receptor-tyrosine kinase inhibitor is at least one selected from the group consisting of gefitinib, erlotinib, osimertinib, afatinib, and sunitinib.

4. The method of claim 2, wherein the subject has not been administered by the epidermal growth factor receptor-tyrosine kinase inhibitor.

5. The method of claim 2, further comprising administering the epidermal growth factor receptor-tyrosine kinase inhibitor to the subject, provided that the expression level of Leucine Zipper Down-regulated in Cancer 1 shows an increased level.

6. The method of claim 2, further comprising providing the subject with an alternative treatment other than administration of the epidermal growth factor receptor-tyrosine kinase inhibitor, provided that the expression level of Leucine Zipper Down-regulated in Cancer 1 shows a decreased level.

7. The method of claim 1, wherein the lung cancer is a non-small cell lung cancer.

8. The method of claim 1, wherein the lung cancer is epidermal growth factor receptor-mutated lung cancer.

9. A kit for predicting drug efficacy of lung cancer in a subject in need thereof, comprising:
an antibody against Leucine Zipper Down-regulated in Cancer 1 or a Leucine Zipper Down-regulated in Cancer 1-specific primer.

10. The kit of claim 9, wherein the drug efficacy is an efficacy of an epidermal growth factor receptor-tyrosine kinase inhibitor.

11. The kit of claim 9, wherein the lung cancer is a non-small cell lung cancer.

12. The kit of claim 9, wherein the lung cancer is epidermal growth factor receptor-mutated lung cancer.

13. A method for treating lung cancer, comprising:
enhancing expression of Leucine Zipper Down-regulated in Cancer 1 encoded by Ldoc1 gene in a subject in need thereof.

14. The method of claim 13, thereby increasing susceptibility of the subject to an epidermal growth factor receptor-tyrosine kinase inhibitor.

15. The method of claim 14, wherein the epidermal growth factor receptor-tyrosine kinase inhibitor is at least one selected from the group consisting of gefitinib, erlotinib, osimertinib, afatinib, and sunitinib.

16. The method of claim 13, further comprising administering the subject with a Leucine Zipper Down-regulated in Cancer 1 protein sequence-derived polypeptide to enhance the Leucine Zipper Down-regulated in Cancer 1 expression in the subject.

17. The method of claim 13, further comprising administering the subject with a Ldoc1 gene sequence-derived nucleotide to enhance the Leucine Zipper Down-regulated in Cancer 1 expression in the subject.

18. The method of claim 13, wherein the lung cancer is a non-small cell lung cancer.

19. The method of claim 13, wherein the lung cancer is epidermal growth factor receptor-mutated.


SEQUENCE LISTING (3 sequences)

SEQ ID NO: 1 - protein, 146 aa, Homo sapiens
MVDELVLLLHALLMRHRALSIENSQLMEQLRLLVCERASLLRQVRPPSCPVPFPETFNGESSRLPEFIVQTASYMLVNENRFCNDAMKVAFLISLLTGEAEEWVVPYIEMDSPILGDYRAFLDEMKQCFGWDDDEDDDDEEEEDDY

SEQ ID NO: 2 - DNA, 58 bp, synthetic construct
  source 1..58: mol_type = other DNA
ccgggctcgtgaacgagaaccgattctcgagaatcggttctcgttcacgagctttttg

SEQ ID NO: 3 - protein, 8 aa, synthetic construct
DYKDDDDK